(19) United States
(12) Patent Application Publication
(10) Pub. No.: US 2021/0393901 A1
(43) Pub. Date: Dec. 23, 2021

(54) SYSTEMS AND METHODS FOR ISOLATING GAS LEAKS

(71) Applicant: Covidien LP, Mansfield, MA (US)

(72) Inventor: Matthew J. Phillips, Carlsbad, CA (US)

(73) Assignee: Covidien LP, Mansfield, MA (US)

(21) Appl. No.: 17/336,441

(22) Filed: Jun. 2, 2021

Related U.S. Application Data
(60) Provisional application No. 63/041,510, filed on Jun. 19, 2020.

(51) Int. Cl. A61M 16/00

(52) U.S. Cl. CPC A61M 16/0087; A61M 2202/0208

(57) ABSTRACT

Aspects of the disclosure describe a design that isolates concentrated oxygen from electrical and/or flammable components of a ventilator. In an example, ventilator components containing or otherwise interacting with concentrated oxygen are isolated from the electrical equipment and/or drained to an exterior of the housing of the ventilator, in case of a leak. For example, an isolating structure encases the concentrated oxygen-carrying components to prevent concentrated oxygen from inadvertently leaking into the inside of the housing of the ventilator. The isolating structure may be a sleeve. An isolation gas inside the isolating structure may be fluidly coupled with ambient air outside the housing of the ventilator to allow the escape of isolation gas outside of the ventilator housing.

### CROSS-REFERENCE TO RELATED APPLICATIONS

This application claims the benefit of U.S. Provisional Application No. 63/041,510, filed Jun. 19, 2020, the complete disclosure of which is hereby incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, and modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

It is with respect to this general technical environment that aspects of the present technology disclosed herein have been contemplated. Furthermore, although a general environment is discussed, it should be understood that the examples described herein should not be limited to the general environment identified herein.

### Systems and Methods for Isolating Gas Leaks

Among other things, aspects of the present disclosure include systems and methods for isolating gas leaks. More specifically, this disclosure describes systems and methods for isolating gas leaks. In an aspect, a ventilator is disclosed. The ventilator includes a housing, the housing comprising an exterior surface and an interior surface. The ventilator also includes an internal oxygen line located inside the housing, the internal oxygen line configured to carry concentrated oxygen at a line pressure. Additionally, the ventilator includes an orifice extending through the housing from the exterior surface to the interior surface. The ventilator further includes an isolation sleeve encasing the internal oxygen line, the isolation sleeve including: a first end; and a second end coupled to the orifice thereby allowing a gas inside the isolation sleeve to escape outside the housing.

In an example, the line pressure is between 15-50 PSI. In another example, the gas inside the isolation sleeve is at atmospheric pressure. In a further example, the internal oxygen line has a first radius and the isolation sleeve has a second radius based on the first radius. In yet another example, a material of the isolation sleeve is silicon. In still a further example, the orifice has a third radius larger than the first radius and is located coaxially with the internal oxygen line. In another example, the oxygen line is fluidly coupled to a ventilator component inside the housing. In a further example, the isolation sleeve also encases the ventilator component. In yet another example, the isolation sleeve comprises a first sleeve segment associated with the oxygen line and a second sleeve segment associated with the ventilator component. In still a further example, the second sleeve segment associated with the ventilator component is coupled to an isolation vent thereby allowing the gas inside the second sleeve segment of the isolation sleeve to escape outside the housing.

In another aspect, a ventilator is disclosed. The ventilator includes a housing comprising an exterior surface and an interior surface. The ventilator further includes an internal oxygen line located inside the housing, the internal oxygen line configured to carry concentrated oxygen at a line pressure. Additionally, the ventilator includes a component located inside the housing, the component fluidly coupled with the oxygen line. The ventilator also includes a first isolation sleeve encasing the internal oxygen line and comprising a first exhaust outside of the housing. Further, the ventilator includes a second isolation sleeve encasing the component and comprising a second exhaust outside of the housing.

In an example, the first exhaust includes a first orifice and the second exhaust comprises a second orifice, wherein the first orifice and the second orifice extend from the exterior surface to the interior surface of the housing. In another example, the first isolation sleeve is coupled to the first orifice. In a further example, the second isolation sleeve is coupled to the second orifice via an isolation vent. In yet another example, the first isolation sleeve and the second isolation sleeve are fluidly coupled. In still a further example, an isolation gas in the first isolation sleeve is isolated from the inside of the housing.

In a further aspect, a ventilator is disclosed. The ventilator includes a housing comprising an exterior surface and an interior surface. Additionally, the ventilator includes an orifice having a first radius and extending through the housing from the exterior surface from the interior surface. The ventilator also includes an oxygen line having a second radius fed through the orifice from the outside of the housing to the inside of the housing. Further, the ventilator includes an isolation sleeve inside the housing coupled to the orifice, the isolation sleeve having the first radius and encasing a portion of the oxygen line existing inside the housing.

In an example, an isolation gas inside the isolation sleeve is fluidly coupled with an outside gas outside the housing, and wherein the isolation gas is isolated from an inside gas inside the housing. In another example, the oxygen line is coupled to an oxygen source. In a further example, the first radius is based on the second radius.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-B show an example isolation sleeve system including an oxygen line and an isolation sleeve.

FIG. 2A shows another example isolation sleeve system including an oxygen line with an isolation sleeve and a ventilator component with an isolation jacket.

FIG. 2B shows yet another example isolation sleeve system including a ventilator component with an isolation jacket and an isolation vent.

FIGS. 3A-B are block diagrams of example isolation systems of a ventilator.

FIG. 4A shows a ventilator with oxygen provided from an oxygen source and air provided from an air source.

FIGS. 4B-D show aspects of the ventilator of FIG. 4A associated with the external oxygen line and external air line.

FIGS. 5A-C show a model of gas pressures and a model of gas velocities for a puncture in the oxygen line and a sleeve puncture in the isolation sleeve.

FIG. 6 is a diagram illustrating an example of a ventilator connected to a human patient.

FIG. 7 is a block-diagram illustrating an example of a ventilatory system.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators provide pressure regulating valves (or regulators) connected to centralized sources of pressurized air and pressurized oxygen (otherwise referred to as an oxygen source and an air source). The regulating valves function to regulate flow so that respiratory gases having a desired concentration are supplied to the patient at desired pressures and flow rates.

While operating a ventilator, it is desirable to control the concentration of oxygen in the gas supplied to the patient. Concentrated oxygen may be provided to the ventilator from an oxygen source. Concentrated oxygen then flows through various components of the ventilator until delivery to the patient. Further, as each patient may require a different ventilation strategy, modern ventilators can be customized for the particular needs of an individual patient.

Use of concentrated oxygen presents an increased risk of fire. As described herein, concentrated oxygen means gas having an oxygen concentration that is greater than that of the ambient air (i.e., gas surrounding the ventilator and existing outside of the housing of the ventilator). For example, various gas lines and components in a ventilator that carry concentrated oxygen may be susceptible to leaks. As concentrated oxygen leaks inside the housing of the ventilator, the materials inside the ventilator may become flammable (i.e., ignite more easily or more rapidly) and may burn at a hotter temperature. For example, a material may have a unique critical oxygen concentration to ignite (e.g., wood is ignitable at a lower critical concentration than plastics). Thus, fire risk is reduced by maintaining an oxygen concentration below the lowest critical concentration of all materials in an environment.

The risk of fire may also be decreased by separating ignition sources, fuel sources, and oxidizer sources. This separation may be applied to components of mechanical ventilators. For example, within a housing of a ventilator, a risk of fire may be reduced by separating concentrated oxygen from electrical components. The isolation of concentrated oxygen from electrical components is encouraged by safety standards. For example, a safety standard that requires separation of concentrated oxygen from electrical components of medical equipment is described by the International Electrotechnical Commission for medical electrical equipment under IEC 60601-1. Thus, separation of concentrated oxygen from electrical components (as is encouraged by safety standards) reduces the risk of fire caused by ventilators. Designs aimed at separating concentrated oxygen from electrical components, however, may result in costly, heavy, large, and/or other non-ideal ventilator designs.

Aspects of this disclosure describe a design that isolates concentrated oxygen from electrical and/or flammable components of a ventilator. In an example, ventilator components containing or otherwise interacting with concentrated oxygen are isolated from the electrical equipment and/or drained to an exterior of the housing of the ventilator, in case of a leak. In an example, an isolation sleeve encasing the concentrated oxygen-carrying components forms an isolating structure so as to prevent concentrated oxygen from inadvertently leaking into the inside of the housing of the ventilator. The isolation sleeve (i.e., isolating structure) may also be in the form of a jacket that may be disposed around non-elongate components. In examples, an isolation sleeve around a non-elongate element may be referred to as a jacket. In an example, the isolation sleeve may be a coaxial sleeve or jacket encasing a component (e.g., oxygen line, valve, accumulator, or any other component that stores, mixes, carries, contains, or is associated with concentrated oxygen inside the housing of the ventilator). With these concepts in mind, several examples of systems and methods for isolating gas leaks are discussed herein.

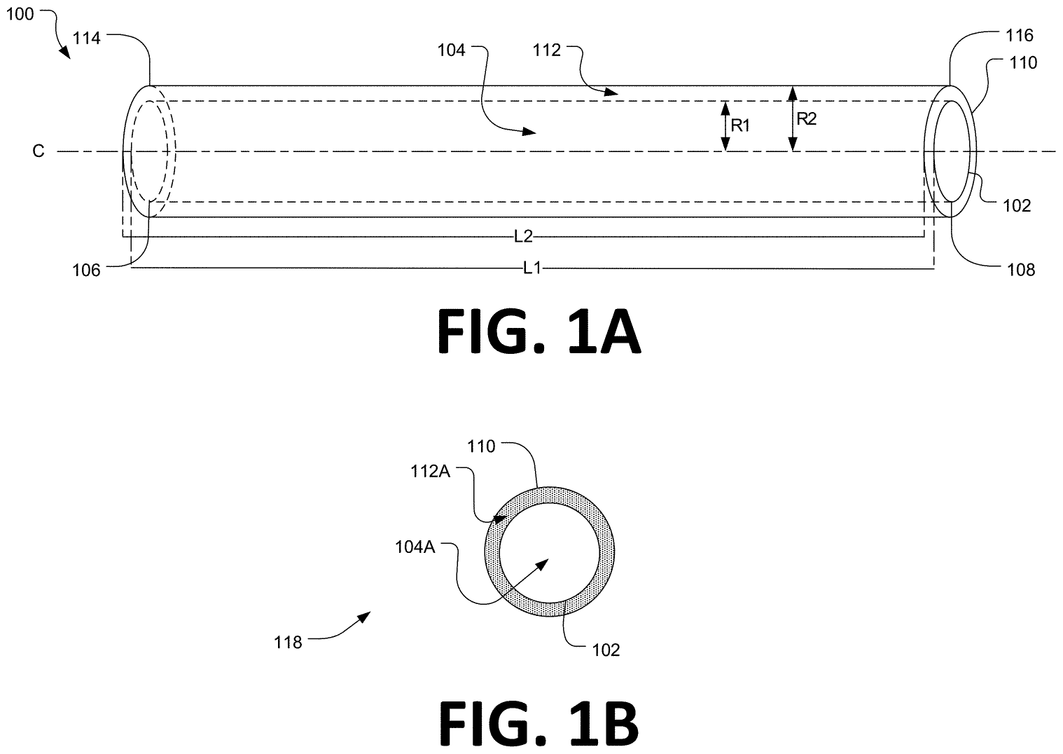

FIGS. 1A-B show an example isolation sleeve system 100 including an oxygen line 102 and an isolation sleeve 110. All or a portion of the isolation sleeve system exists inside of a housing of a ventilator. In the example shown, the oxygen line 102 contains concentrated oxygen. The concentrated oxygen in the oxygen line 102 (i.e., the line pressure) is pressurized above ambient pressure. The line pressure may be at or below a pressure of the oxygen source. In an example, the oxygen source may be outside of the housing of the ventilator or may be concentrated at or inside of the ventilator. In an example, the concentrated oxygen may be supplied from a wall of a hospital, a bottled source, a concentrator, etc. and may be pressurized above ambient pressure. In another example, the source pressure may be 50 PSI. The oxygen pressure may be reduced prior to entering the ventilator system. For example, the pressure of the oxygen gas may be reduced from 50 PSI to 20 PSI prior to entering the ventilator housing, or at or near the ventilator housing. Thus, the pressure of concentrated oxygen in the oxygen line (i.e., the line pressure) may be above atmospheric pressure and below a source pressure. For example, the line pressure may be 30 PSI. The oxygen line 102 may include one or more materials or compounds capable of maintaining integrity while containing the line pressure. For example, the oxygen line 102 may be made of a flexible tubing material such as silicone rubber, silicon, high-density polyethylene, thermoplastic copolymer, rubber-styrenic block copolymer, thermoplastic elastomer, etc. Alternatively, the oxygen line 102 may be made of a rigid tubing material such as copper, aluminum alloy, steel, polycarbonate, Delrin, Nylon, PVC, etc.

In another example, the oxygen line 102 may be tubular in shape, such that a line volume 104 of the oxygen line 102 is defined by a line radius R1, a first end 106, and a second end 108 of the oxygen line 102. A length L1 of the oxygen line 102 is defined as the distance between the first end 106 and the second end 108 of the oxygen line 102 along a center axis C of the oxygen line 102. In another example, the oxygen line 102 may be non-tubular. The oxygen gas in the oxygen line 102 flows from the first end 106 to the second end 108 of the oxygen line 102. The first end 106 and the second end 108 of the oxygen line 102 may be coupled to different components. In an example, the first end may be coupled to an oxygen line joint near the housing of the ventilator. The oxygen line joint may couple the oxygen source with the oxygen line to allow the flow of concentrated oxygen into the ventilator. Alternatively, the first end 106 of the oxygen line 102 may be coupled to a valve (e.g., to reduce the line pressure from the source pressure of concentrated oxygen). In another example, the first end 106 of the oxygen line may be otherwise removably coupled to the oxygen source.

All or a portion of the oxygen line 102 exists inside a housing of the ventilator. In an example, the oxygen line is fed through the housing of the ventilator to carry concentrated oxygen from the oxygen source into the housing of the ventilator. The oxygen line 102 may have one or more non-consecutive segments inside the housing of the ventilator to carry concentrated oxygen to various components inside the housing before delivering concentrated oxygen to a patient.

As shown in FIGS. 1A-B, the isolation sleeve 110 has a first end 114 and a second end 116. A length L2 of the isolation sleeve 110 is defined as the distance between the first end 114 and the second end 116 of the isolation sleeve 110 along a center axis C of the isolation sleeve 110. The center axis C of the isolation sleeve 110 may be different from the center line C of the oxygen line 102. In an example, the isolation sleeve 110 encases all or a portion of the oxygen line 102 inside the housing of the ventilator. For example, the first end 114 of the isolation sleeve 110 and the first end 106 of the oxygen line 102 may exist along a common cross section 118 perpendicular to a shared center axis C, or may each exist along a different cross section 118 along the shared center axis C. Similarly, the second end 116 of the isolation sleeve 110 and the second end 108 of the oxygen line 102 may exist along a common cross section 118, or may each exist along a different cross section 118 along the center axis C. In another example, the length L1 of the oxygen line 102 may be different than the length L2 of the isolation sleeve 110. For example, the first end 106 and/or the second end 108 of the oxygen line 102 may be coupled with a ventilator component (e.g., valve, accumulator, joint, connection, etc.). If the oxygen line 102 is coupled to a ventilator component, then the first end 114 and/or the second end 116 of the isolation sleeve 110 may terminate at or before the ventilator component. Alternatively, the isolation sleeve 110 may terminate (i.e., have a first end 114 or a second end 116) at another isolation sleeve associated with the ventilator component, or may continue to encase all or a portion of the ventilator component (e.g., extend past either the first end 106 or the second end 108 of the oxygen line 102).

The isolation gas in the isolation sleeve 110 may be in fluid communication with the ambient air, or may otherwise allow isolation gas inside the isolation sleeve 110 to escape the housing of the ventilator (e.g., the first end 114 or the second end 116 of the isolation sleeve 110 may be open to ambient). In an example where the integrity of the oxygen line is maintained (e.g., no punctures, rips, defects, etc. such that the gas in the oxygen line 102 is separate from the gas in the isolation sleeve 110), the pressure and composition of the isolation gas in the isolation sleeve 110 is the same as ambient. In an example where there is a leak in the oxygen line 102 into the isolation sleeve 110 and the integrity of the isolation sleeve 110 is maintained, concentrated oxygen flows into the isolation sleeve and then ultimately flows outside of the housing of the ventilator without leaking into the inside of the housing.

In another example, both the isolation sleeve 110 and the oxygen line 102 may be tubular in shape, such that an isolation volume 112 of the isolation sleeve 110 is defined between a sleeve radius R2 (i.e., an outer radius), the line volume 104 of the oxygen line 102 (e.g., as may be based on the line radius R1, first end 106 and second end 108 of the oxygen line 102), a first end 114 of the isolation sleeve 110, and a second end 116 of the isolation sleeve 110. In the example shown, the isolation sleeve 110 and the oxygen line 102 are parallel, coaxial tubes. In another example, the isolation sleeve 110 and/or the oxygen line 102 may be non-tubular. The isolation sleeve 110 may not be in contact with the oxygen line 102 along one or more portions of the oxygen line 102. For example, the oxygen line 102 may be nested inside the isolation sleeve 110 without contacting the isolation sleeve 110 such that the isolation volume 112 completely encases the oxygen line 102, as shown in the cross section 118 perpendicular to the oxygen line 102 between the first end 106 and the second end 108 in FIG. 1B. Alternatively, the oxygen line 102 may be off-center or non-coaxial with the isolation sleeve 110 (e.g., the oxygen line 102 may have a different center line than the isolation sleeve 110) In an example, one or more points along the external surface of the oxygen line 102 may be in contact with the internal surface of the isolation sleeve 110, such that the oxygen line 102 is fully encased by the isolation sleeve 110 but the isolation volume 112 does not extend continuously around the oxygen line 102. In another example, one or more portions of the oxygen line 102 may be encased by at least one isolation sleeve 110. In a further example, one or more portions of the oxygen line 102 may not be encased by the isolation sleeve 110. For example, a portion of an oxygen line 102 that is less susceptible to leaks or is unlikely to leak may not be encased by the isolation sleeve 110.

At a cross section 118 of the isolation sleeve system 100, the line volume 104 is based on a cross-sectional line area 104A (i.e., the area bounded by the oxygen line 102 at the cross section 118) and the isolation volume 112 is based on a cross-sectional isolation area 112A (i.e., the annulus bounded between the external wall of the oxygen line 102 and the internal wall of the isolation sleeve 110 at the cross section 118). A ratio between the cross-sectional isolation area 112A and the cross-sectional line area 104A may be based on resistance of gas flow through the isolation volume 112 (e.g., pipe loss, surface roughness, etc.), material of the oxygen line 102 and/or the isolation sleeve 110, pressure of the oxygen line 102, pressure difference between the pressure in the oxygen line 102 and ambient pressure, oxygen concentration of gas in the oxygen line 102, flowrate of gas in the oxygen line, radius R1 of the oxygen line, radius R2 of the isolation sleeve, quantity of isolation orifices (e.g., outlets or drains where the isolation gas inside the isolation sleeve is fluidly coupled with ambient), or any other factor that may affect or impact escape of isolation gas in the isolation sleeve 110 outside the housing of the ventilator through one or more orifices (e.g., as may be coupled to first end 114 and/or second end 116 of the isolation sleeve 110). Additionally or alternatively, relative sizing of one or more dimensions of the oxygen line 102 and/or the isolation sleeve 110 (e.g., radius R1, radius R2) may be based on a ratio. For example, the ratio may be based on the radius R1 of the oxygen line, radius R2 of the isolation sleeve 110, length of the oxygen line L1, length of the isolation sleeve L2, volume of the oxygen line 102, volume of the isolation sleeve 110, pressure of gas in the oxygen line 102, or any other factor related to flow of gas in the isolation volume 112, in any combination.

The isolation sleeve 110 may include one or more materials or compounds that may be a range of flexibilities (e.g., inflatable, semi-rigid, rigid, etc.). For example, the isolation sleeve 110 may be flexible tubing having a material such as silicon, silicone rubber, high-density polyethylene, thermoplastic copolymer, rubber-styrenic block copolymer, thermoplastic elastomer, etc. In another example, the isolation sleeve 110 is capable of maintaining a tubular shape and being concentric with the oxygen line 102. The isolation sleeve 110 may be the same material as the oxygen line 102.

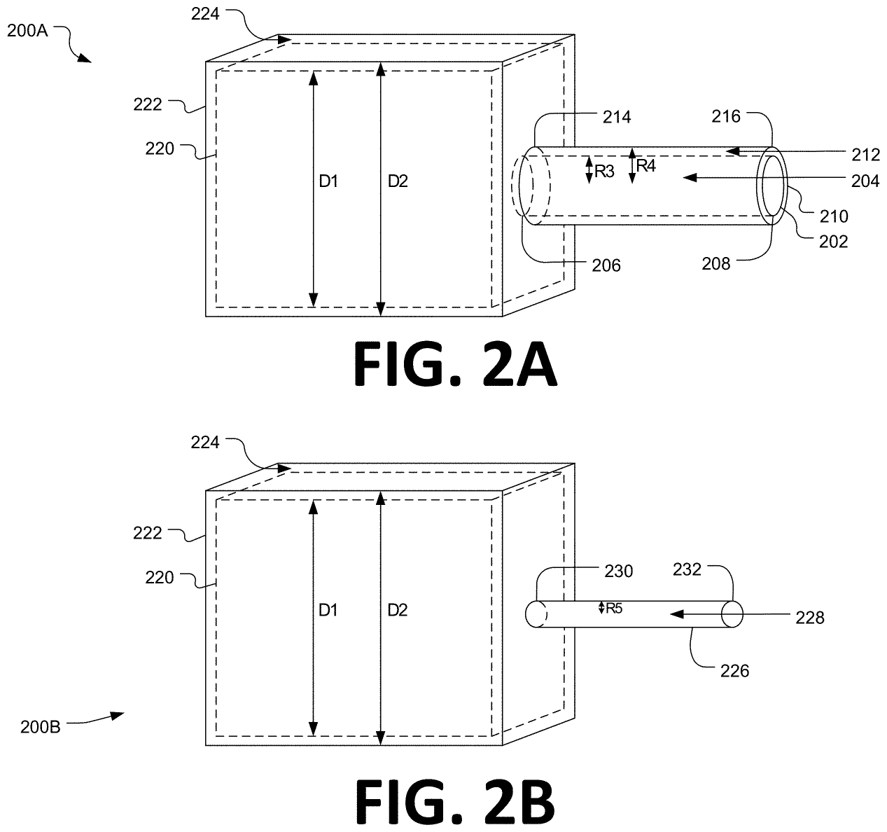

FIG. 2A shows another example isolation sleeve system 200A including an oxygen line 202 with an isolation sleeve 210, and a ventilator component 220 with an isolation jacket 222 (i.e., an isolation sleeve about a non-elongate component, or an isolating structure). Aspects of the isolation sleeve system 200A may be similar to isolation sleeve system 100 in FIGS. 1A-B. For example, aspects of the oxygen line 202 and isolation sleeve 210 may be similar to oxygen line 102 and isolation sleeve 110, respectively. The oxygen line 202 may have a first end 206, a second end 208, a radius R3, and a line volume 204. The isolation sleeve 210 associated with the oxygen line 202 may have a first end 214, a second end 216, a radius R4, and an isolation volume 212. The first end 206 and/or second end 208 of the oxygen line 202 may be coupled to a ventilator component 220, as described herein. In an example, the first end 206 of the oxygen line 202 is coupled to the ventilator component 220. The coupling of the oxygen line 202 to the ventilator component 220 may allow the flow of concentrated oxygen, which may be pressurized, contained in the oxygen line 202 into the ventilator component 220. Additionally or alternatively, the coupling of the oxygen line 202 to the ventilator component 220 may allow the flow of the concentrated oxygen contained in the ventilator component 220 into the oxygen line 202. For example, the concentrated oxygen may flow from the oxygen line 202 into the ventilator component 220, or may flow from the ventilator component 220 into the oxygen line 202.

The ventilator component 220 may have an associated isolation jacket 222 with an isolation volume 224. The isolation volume 224 may be defined by the space existing between the isolation jacket 222 and the ventilator component 220. In an example, the ventilator component 220 may have at least one component dimension D1 and the associated isolation jacket 222 may have at least one isolation dimension D2. A component dimension D1 may include a length, width, height, radius, volume, surface area, or any other measurement associated with an internal or external feature of the component. The isolation volume 224 may be based on the component dimension D1 and/or the isolation dimension D2. An isolation dimension D2 may include a length, width, height, radius, volume, surface area, or other measurement associated with the isolation jacket 222.

Sizing of the isolation jacket 222 associated with the ventilator component 220 may be based on one or more aspects of the isolation sleeve system 200A. For example, the size, shape, or configuration of the isolation jacket 222 may be based on the radius R3 of the oxygen line, radius R4 of the isolation sleeve 110, volume of the oxygen line 102, volume of the isolation sleeve 110, component dimension D1, sleeve dimension D2, isolation volume 224, volume and/or pressure of concentrated oxygen in the ventilator component 220, quantity of isolation orifices (e.g., amount of orifices at which the gas in the isolation volume 224 is in fluid communication with ambient), length of the oxygen line 202, length of the isolation sleeve 210, or any other factor related to flow of gas in the isolation volume 224, in in any combination.

In an example where the isolation sleeve system 200A includes at least one oxygen line 202 with an associated isolation sleeve 210 and at least one ventilator component 220 with an associated isolation jacket 222, the isolation sleeves 210, 222 may have multiple configurations. For example, the gas in the isolation sleeve 210 of the oxygen line may be in fluid communication with the gas in the isolation jacket 222 of the ventilator component. For example, the isolation sleeves 210, 222 may be coupled at the first end 214 of the isolation sleeve 210 of the oxygen line 202. The size and/or shape and/or configuration of the isolation sleeves 210, 222 may vary based on whether the gas of the isolation sleeves 210, 222 is in fluid communication (e.g., the isolation sleeve 210 of the oxygen line 202 enables the escape of gas in the isolation jacket 222 of the ventilator component 220 outside the housing of the ventilator). Alternatively, the isolation sleeves 210, 222 may be separated or isolated such that gas in the isolation sleeve 210 associated with the oxygen line 202 is not in fluid communication with the gas in the isolation jacket 222 associated with the ventilator component 220.

FIG. 2B shows yet another example isolation sleeve system 200B including a ventilator component 220 with an isolation jacket 222 and an isolation vent 226. In an example, the isolation jacket 222 of a ventilator component 220 may have an associated isolation vent 226 to allow gas inside the isolation volume 224 to exit the housing of the ventilator. For example, the isolation vent may have a first end 230 coupled to the isolation jacket 222 to allow gas inside the isolation jacket 222 to be in fluid communication with the gas inside the isolation vent 226. The isolation vent 226 may have a second end 232 coupled to an orifice to allow gases inside the isolation vent 226 to be in fluid communication with ambient. Thus, the isolation vent 226 may allow or enable the escape of gases in the isolation jacket 222 and/or isolation vent 226 to escape to outside of the housing of the ventilator. The isolation vent 226 may have a vent volume 228 defined by the first end 230, second end 232, and a vent radius R5 of the isolation vent 226.

In an example, the isolation sleeve system 220A or 200B may include both an isolation vent 226 and an isolation sleeve 210 associated with an oxygen line 202. In this example, one or more of the following gases may be in fluid communication with each other: the gases in the isolation sleeve 210 associated with the oxygen line 202; gases in the isolation jacket 222 associated with the ventilator component 220; and/or gases in the isolation vent 226. In another example, the isolation sleeve 210 associated with the oxygen line 202, the isolation jacket 222 associated with the ventilator component 220, and/or the isolation vent 226 may be coupled to an orifice (e.g., to allow gases the in isolation sleeves 210, 222, and/or isolation vent 226 to escape outside the housing of the ventilator).

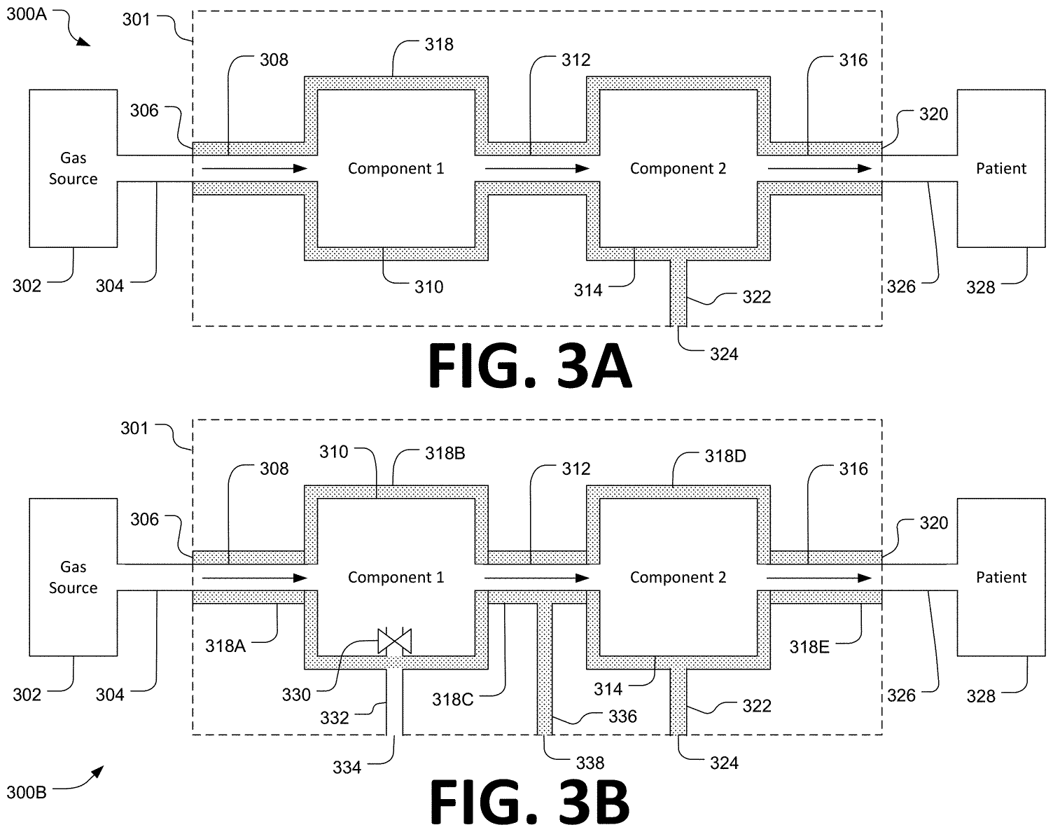

FIGS. 3A-B are block diagrams of example isolation systems 300A, 300B of a ventilator 301. In an example, gas may flow from a gas source 302 to a ventilator 301 to a patient 328. The gas source 302 may be located outside of the housing of the ventilator 301. In an example, gas is provided to the ventilator 301 from the gas source 302 via an external gas line 304, external to the housing of the ventilator 301. The external gas line 304 may couple to an internal gas line (e.g., first gas line 308) at a gas line joint, to allow the flow of gas from the gas source 302 into the ventilator 301.

For example, gas may enter the housing of the ventilator 301 at a first gas line 308. The first gas line 308 may allow gas to flow to a first component 310 inside the housing of the ventilator 301. Gas may then flow from the first component 310 to a second gas line 312. Gas may then flow from the second gas line 312 to a second component 314. Gas may then flow from the second component 314 to a third gas line 316. The third gas line 316 may provide the gas through an inhalation limb 328 outside the housing of the ventilator 301 to be delivered to a patient 328. Although FIGS. 3A-B show three gas lines (i.e., first gas line 308, second gas line 312, and third gas line 316) and two components (i.e., first component 310 and second component 314), it should be appreciated that any quantity of gas lines and/or components may be located inside the housing of the ventilator 301, in any order and/or combination. The pressure of the gas in a gas line (e.g., first gas line 308, second gas line 312, third gas line 316) (otherwise referred to herein as a line pressure) and/or a pressure of the gas in a component (e.g., first component 310, second component 314), may vary within the gas line and/or component or vary from line-to-line and/or component-to-component. For example, the pressure of the gas in the first gas line 308 may differ from the pressure of the gas in the first component 310, which may differ from the pressure of the gas in the second gas line 312, which may differ from the pressure in the second component 314, which may differ from the pressure of the gas in the third gas line 316). In an example, the pressure of the gas in a gas line and/or in a component is at or above atmospheric pressure (e.g., ambient pressure).

In an example, the gas source may provide pressurized, concentrated oxygen. The gas delivery may start with the ventilator connected to an air and/or oxygen source. Gas may travel from the source to a mix module where gas pressures are regulated by their respective proportional solenoid valve (otherwise referred to as a “PSOL”) (e.g., a first component 310). One or more PSOLs meter the gases according to the ventilator settings, then the gases may flow through individual air and/or oxygen flow sensors into the mix manifold and accumulator for mixing (e.g., second component 314). The individual gas pressures are continuously monitored both before and after they are mixed in the mix manifold and accumulator assemblies. The mixed gas may then flow to the inspiratory pneumatic system where it flows through the breath delivery flow sensor (e.g., another component) and then the inspiratory PSOL (e.g., another component) for delivery to the patient 328.

In an example, the ventilator 301 may include an isolation sleeve 318 inside the housing of the ventilator 301. The isolation sleeve 318 may isolate one or more gas lines and/or components from other portions (e.g., electrical components) of the ventilator 301. The isolation sleeve 318 may be coupled to a first orifice 306. The first orifice 306 may extend through the housing of the ventilator 301 such that the gas in the isolation sleeve 318 is fluidly coupled with gas outside the ventilator 301, but not fluidly coupled with gas inside the housing of the ventilator 301 (e.g., gas in the isolation sleeve 318 is isolated from gas in the inside of the housing of the ventilator 301). The first orifice 306 may have any predefined shape. For example, the first orifice may be the same shape as a portion of the isolation sleeve 318. Alternatively, the first orifice 306 may have a different shape than the isolation sleeve 318 with an appropriate transition from the first orifice 306 to the isolation sleeve 318. The isolation sleeve 318 may additionally be coupled to a second orifice 320. The second orifice 320 may be similar to the first orifice 306.

In the example shown in FIG. 3A, the isolation sleeve 318 encases multiple gas lines and multiple components. For example, a single or continuous isolation sleeve 318 may encase one or more of: the first gas line 308; first component 310; second gas line 312; second component 314; and third gas line 316. In this example, if any of the gas lines and/or gas components were to leak gas into the isolation sleeve 318, the single, continuous isolation sleeve 318 may allow gas in the isolation sleeve 318 to escape through one or more orifices 306, 320. In an example, an isolation vent 322 (i.e., a line fluidly coupled to the isolation sleeve 318 that does not surround a gas line or component) may be connected to another orifice 324, to allow gas in the isolation sleeve 318 to escape the housing of the ventilator 301. In an example, an isolation vent 322 may be located proximate or adjacent to a component (e.g., as shown proximate to the second component 314). In an example, an isolation vent 322 may be adjacent to a gas line or a component that is distant from another orifice (e.g., orifice 306, 320, 324) coupled to the isolation sleeve 318 to promote escape of gases outside the housing of the ventilator 301. In another example, the isolation sleeve 318 may be coupled to multiple isolation vents.

In the example shown in FIG. 3B, the ventilator 301 may include multiple isolation sleeve segments 318A-E. Each isolation sleeve segment 318A-E may be associated with one or more gas lines and/or components of the ventilator 301. In an example, the first gas line 308 may be associated with a first isolation sleeve segment 318A. In another example, the first component may be associated with a second isolation sleeve segment 318B. In a further example, the second gas line 312 may be associated with a third isolation sleeve segment 318C. In another example, the second component 314 may be associated with a fourth isolation sleeve segment 318D. In a further example, the third gas line 316 may be associated with a fifth isolation sleeve segment 318E. The isolation sleeve segments 318A-E may be contiguous.

Each of the isolation sleeve segments 318A-E may be coupled to an orifice (e.g., orifices 306, 320, 324, 334, 338) and/or coupled to an isolation vent (e.g., isolation vents 322, 336) and/or coupled to an isolation bypass (e.g., isolation bypass 332). In an example, an isolation sleeve associated with a gas line may be coupled to an orifice at an end of the isolation sleeve segment (e.g., first gas line 308 associated with isolation sleeve segment 318A coupled to orifice 306, or third gas line 316 associated with fifth isolation sleeve segment 318E coupled to orifice 320). In another example, an isolation sleeve associated with a gas line may be coupled to an orifice via an isolation vent to allow gas in the isolation sleeve to escape the housing of the ventilator 301 (e.g., second gas line 312 associated with third isolation sleeve segment 318C coupled to isolation vent 336 coupled to orifice 338). In a further example, an isolation sleeve segment associated with a component may be coupled to an orifice via an isolation vent (e.g., second component 314 associated with fourth isolation sleeve segment 318D coupled to isolation vent 322 coupled to orifice 324).

In another example, an isolation sleeve segment associated with a component may be coupled to an orifice via an isolation bypass (e.g., first component 310 associated with isolation sleeve segment 318B coupled to isolation bypass 332 coupled to orifice 334). An isolation bypass (e.g., isolation bypass 332) may be coupled to an orifice (e.g., orifice 334) and one or more of a component (e.g., first component 310) or an associated isolation sleeve (e.g., isolation sleeve segment 318B). The isolation bypass 332 may have a valve 330 to control the amount of gas exiting the first component 310 and/or the associated isolation sleeve segment 318B into the isolation bypass 332 to escape the ventilator housing through the orifice 334. In an example, the isolation sleeve 318 may drain directly into the isolation bypass 332. In another example, the valve 330 may control the amount of gas in the isolation sleeve entering the isolation bypass 332. In a further example, the first component 310 may be directly coupled to the isolation bypass 332 controlled by valve 330.

In a further example, the isolation system 300A, 300B may include an exit plenum located inside the housing of the ventilator 301. The exit plenum may be an isolated space inside the housing of the ventilator 301 fluidly coupled to ambient air to passively or actively allow plenum gas inside the exit plenum to escape the housing of the ventilator 301. In an example, one or more isolation sleeve segments may be in fluid communication with the isolated plenum to shunt isolation gas inside the isolation sleeve segment into the exit plenum to then escape the housing of the ventilator 301.

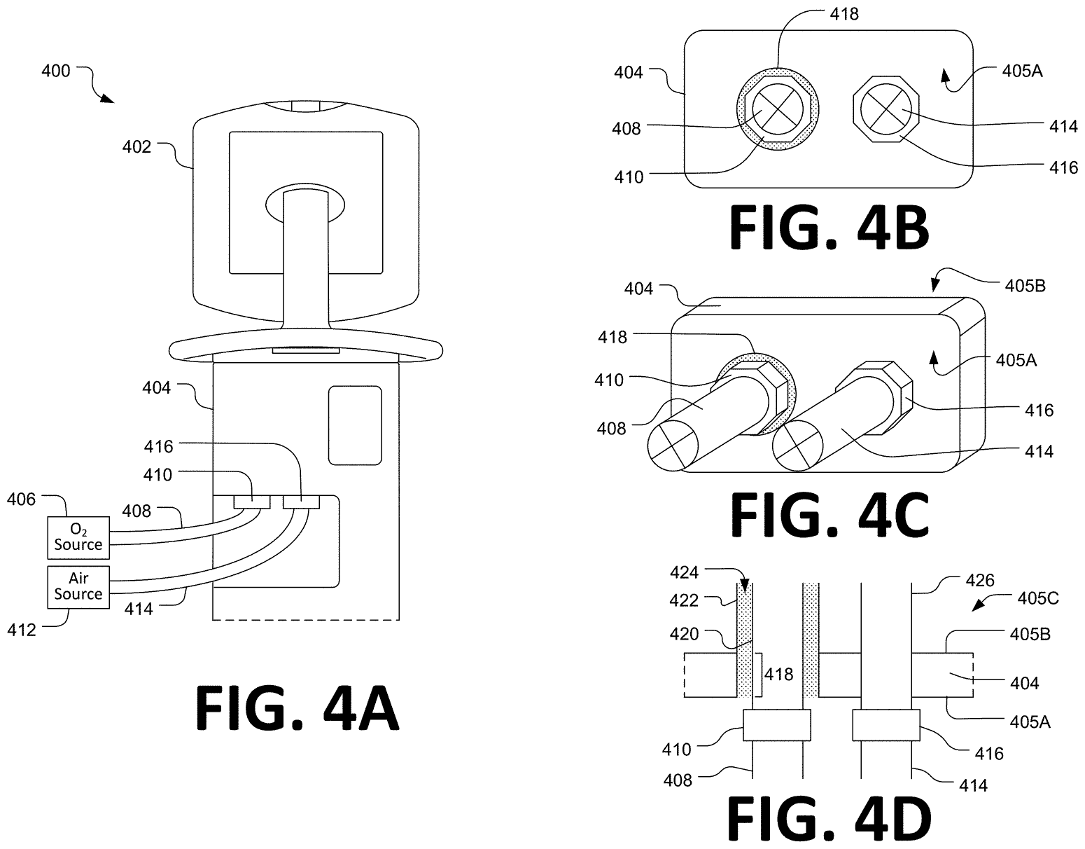

FIG. 4A shows a ventilator 400 with concentrated oxygen provided from an oxygen source 406 and air provided from an air source 412. The ventilator 400 may have a display 402 and a housing 404. The housing 404 may encase components of the ventilator 400 (e.g., components are inside the housing 404 of the ventilator 400), as described herein. The housing 404 may be a rigid or semi-rigid material encasing components associated with the ventilator 400. The oxygen source 406 may be outside the housing 404 of the ventilator 400 and may provide concentrated oxygen to the ventilator 400 through an external oxygen line 408, which may couple to an internal oxygen line 420 at an oxygen line joint 410. The oxygen line joint 410 may allow coupling of the external oxygen line 408 to the internal oxygen line 420 to allow the flow of oxygen from the oxygen source 406 into the ventilator 400. Similarly, the air source 412 may be outside the housing 404 of the ventilator 400 and may provide air to the ventilator 400 through an external air line 414 which may couple to an internal air line 426 at an air line joint 416. The air line joint 416 may allow coupling of the external air line 414 with the internal air line 426 to allow the flow of air from the air source 412 into the ventilator 400.

FIGS. 4B-D show portions of the ventilator of FIG. 4A associated with the external oxygen line 408 and external air line 414. For example, FIGS. 4B and 4C show different perspectives from the outside of the housing 404 of the ventilator 400, including the oxygen line joint 410 and the air line joint 416. FIG. 4D shows a cross-sectional view of the housing 404 at the oxygen line joint 410 and the air line joint 416. The housing 404 has an exterior surface 405A facing the outside of the ventilator 400 and an interior surface 405B facing the inside of the ventilator 400. The external oxygen line 408 may carry concentrated oxygen from the oxygen source 406 to the ventilator 400. The concentrated oxygen may flow into an internal oxygen line 420, at least a portion of which is located inside the housing 404 of the ventilator 400. A portion of the external oxygen line 408 and/or the internal oxygen line 420 may extend through an orifice 418 in the housing 404 (e.g., an oxygen line may be fed through the orifice 418 to carry concentrated oxygen into the housing 404 of the ventilator 400). The orifice 418 extends through the housing 404 and may be a variety of shapes. For example, the orifice 418 may have any predefined shape. For example, the orifice 418 may be the same shape as a portion of the isolation sleeve 422 or may have a different shape than the isolation sleeve 422 with an appropriate transition from the orifice 418 to the isolation sleeve 422.

Inside of the housing 404, an isolation sleeve 422 may encase a portion of the internal oxygen line 420 and couple to the orifice 428. The isolation sleeve 422 may have an isolation volume 424, as described herein. As further described herein, the isolation gas in the isolation volume 424 is fluidly coupled with ambient air via the orifice 418, which extends through the housing 404 to the exterior surface 405A.

The external air line 414 may carry air from the air source 412 to the ventilator 400. The air may flow into an internal air line 426, at least a portion of which is located inside the housing 404 of the ventilator 400. A portion of the external air line 414 and/or the internal air line 426 may extend through the housing 404 (e.g., an air line may be fed through the housing 404). The internal air line 426 may not be associated with an isolation sleeve 422 and/or an orifice 418.

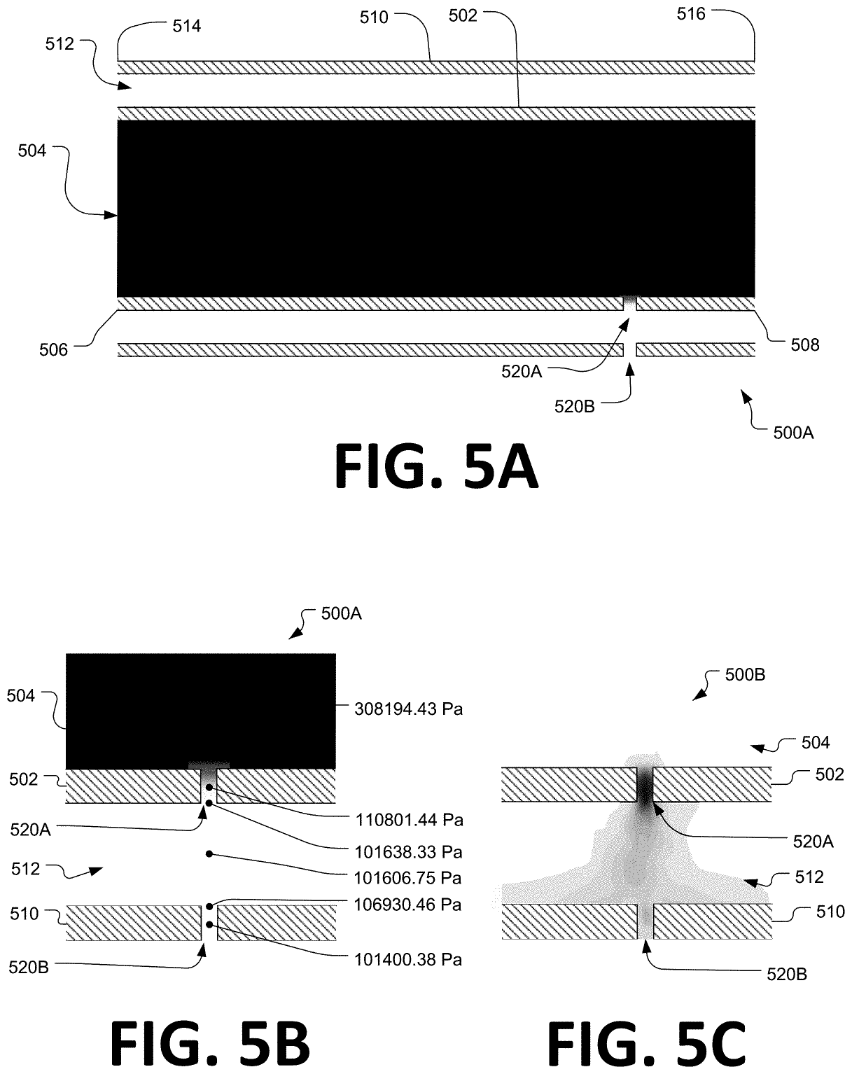

FIGS. 5A-C show a model of gas pressures 500A and a model of gas velocities 500B for a line puncture 520A in the oxygen line 502 and a sleeve puncture 520B in the isolation sleeve 510. The oxygen line 502 may have similar features to gas lines described herein (e.g., oxygen line 102, oxygen line 202, first gas line 308, second gas line 312, third gas line 316, internal oxygen line 420, etc.). The isolation sleeve 510 may have similar features to isolation sleeves described herein (e.g., isolation sleeve 110, isolation sleeve 210, isolation sleeve 318, isolation sleeve 422, etc.). For example, the oxygen line 502 may have a first end 506 and a second end 508 with a line volume 504. As another example, the isolation sleeve 510 may have a first end 514 and a second end 516 with a sleeve volume 512. In an example, the gas in the oxygen line 502 may be pressurized (e.g., above atmospheric pressure) and isolation gas in the isolation sleeve 510 may be at atmospheric pressure, prior to a leak (e.g., at line puncture 520A and/or at sleeve puncture 520B). For example, the gas in the oxygen line 502 may be at 30 PSI.

In the example gas pressure model 500A shown in FIGS. 5A-B and the example gas velocity model 500B shown in FIG. 5C, a leak assessment was approximated and modeled for an oxygen line 502 encased by a coaxial isolation sleeve 510, each with 0.5 mm diameter punctures (i.e., a hole to allow leak). The line puncture 520A and the sleeve puncture 520B may be coaxially aligned at both the oxygen line 502 and the isolation sleeve 510 (i.e., an aligned double failure of the system, or a double leak or a leak in both the oxygen line 502 and the isolation sleeve 510). The gas pressure model 500A shows estimated pressures of the concentrated gas in the oxygen line 502 as it leaks out into the isolation sleeve 510. A darker color indicates a higher pressure of the concentrated gas in the oxygen line 502. Lighter colors in the isolation sleeve 510 indicate that the gas inside the isolation sleeve is at a lower pressure. The gas pressure model 500A depicts pressure estimations of the gas in the oxygen line 502 leaking into the isolation sleeve 510 at steady state.

In the example shown in FIGS. 5A-B, the pressure of the gas is variable. For example, as shown, the pressure of gas in the line volume 504 of the oxygen line 502 is at approximately 308.194 kPa (i.e., approximately 44.70 PSI). The pressure decreases along line puncture 520A. As shown, a pressure inside the line puncture 520A is approximately 110.801 kPa (i.e., approximately 16.07 PSI). Additionally, as shown, the pressure of the gas at the junction of the line puncture 520A and the isolation volume 512 is approximately 101.638 kPa (i.e., approximately 14.74 PSI). A pressure of the gas in the isolation volume 512, as shown, is approximately 101.607 kPa (i.e., approximately 14.74 PSI). As also shown, the pressure of the gas at a junction of the isolation volume 512 and the sleeve puncture 520B is approximately 106.930 kPa (i.e., approximately 15.51 PSI). As also shown, the pressure of the gas inside the sleeve puncture 520B is approximately 101.400 kPa (i.e., approximately 14.71 PSI). In FIGS. 5A-C, atmospheric pressure (i.e., pressure of gas outside of the isolation sleeve and pressure of gas outside the housing of the ventilator) is assumed to be approximately 101.325 kPa (i.e., approximately 14.70 PSI). The pressure models depicted in FIGS. 5A-5C are provided merely as an example. As should be appreciated, different configurations and puncture types may result in different pressure distributions.

The gas velocity model 500B shown in FIG. 5C shows estimated velocities of the gas in the oxygen line 502 as it leaks into the isolation sleeve 510. A darker color indicates a higher velocity of gas. Lighter colors indicate lower velocity. As shown, the gas in the line puncture 520A have a higher velocity. The velocity of the gas drops as it enters the isolation sleeve 510 but maintains a higher velocity than gases in other portions of the isolation sleeve 510. Additionally, as shown, the velocity of gas in the sleeve puncture 520B is also higher than velocity of gases in other portions of the isolation sleeve 510. The gas velocity model 500B depicts velocity estimations of the gas in the oxygen line 502 leaking into the isolation sleeve 510 at the same steady state as in gas pressure model 500A in FIGS. 5A-B.

In the example shown in FIGS. 5A-B, the concentrated gas in the oxygen line 502 leaks out of the oxygen line 502 at line puncture 520A. The concentrated gas leaks into the isolation sleeve 510 at line puncture 520A. When the concentrated gas leaks into the isolation sleeve 510, the concentrated gas mixes with the isolation gas inside of the isolation sleeve 510. The composition of the mixed gas in the isolation sleeve 510 varies along the length of the isolation sleeve (e.g., sleeve length L2), approaching ambient composition at an orifice coupled to the isolation sleeve 510 and approaching the composition of the concentrated oxygen in the oxygen line 502 at the line puncture 520A. The pressure of the mixed gas in the isolation sleeve 510 varies along the length of the isolation sleeve 510, resulting in a pressure difference along the length of the isolation sleeve 510. The pressure difference along the length of the isolation sleeve results in a flow of the mixed gas in the isolation sleeve 510 outside the housing of the ventilator at the orifice open to ambient pressure. Thus, concentrated oxygen leaking into the isolation sleeve 510 from the oxygen line 502 may escape the housing of the ventilator through an orifice open to ambient.

As further shown in FIGS. 5A-B, the mixed gas in the isolation sleeve 510 leaks out of the isolation sleeve 510 into the inside of the ventilator at the sleeve puncture 520B. In this example, the line puncture 520A in the oxygen line 502 and the sleeve puncture 520B in the isolation sleeve 510 are directly aligned. As calculated in the example shown, less than about 20% or 25% of the concentrated gas from the oxygen line 502 is estimated to leak through the sleeve puncture 520B into the inside of the ventilator housing for this configuration and puncture size and configuration. Likewise, based on the model in FIG. 5A-C, 75% or more of the concentrated gas flowing through the line puncture 520 from the oxygen line 502 is captured or maintained inside of the isolation volume 512 and may escape outside an orifice. In some calculations 75-80% of the concentration gas leaving the oxygen line 502 is captured inside the isolation volume 512 rather than leaking out of the sleeve puncture 520B. In some calculations, greater than 80% of the concentration gas leaving the oxygen line 502 is captured inside the isolation volume 512. Accordingly, a significant amount of oxygen is prevented from entering the ventilator and may be evacuated outside the ventilator through an orifice as discussed above.

In an example where the punctures are not aligned, the leak into the inside of the ventilator housing may be less. In an example where there is only a single failure (e.g., a leak in the oxygen line 502, but not the isolation sleeve 510; or a leak in the isolation sleeve 510, but not the oxygen line 502), no gas from the oxygen line 502 will leak into the inside of the ventilator housing (i.e., the concentrated gas will be completely isolated from the space on the inside of the ventilator housing). Thus, in an example where there is a leak in the oxygen line 502 inside of a ventilator housing, the amount of concentrated gas leaking inside the ventilator housing may be prevented or reduced with the isolation sleeve systems described herein.

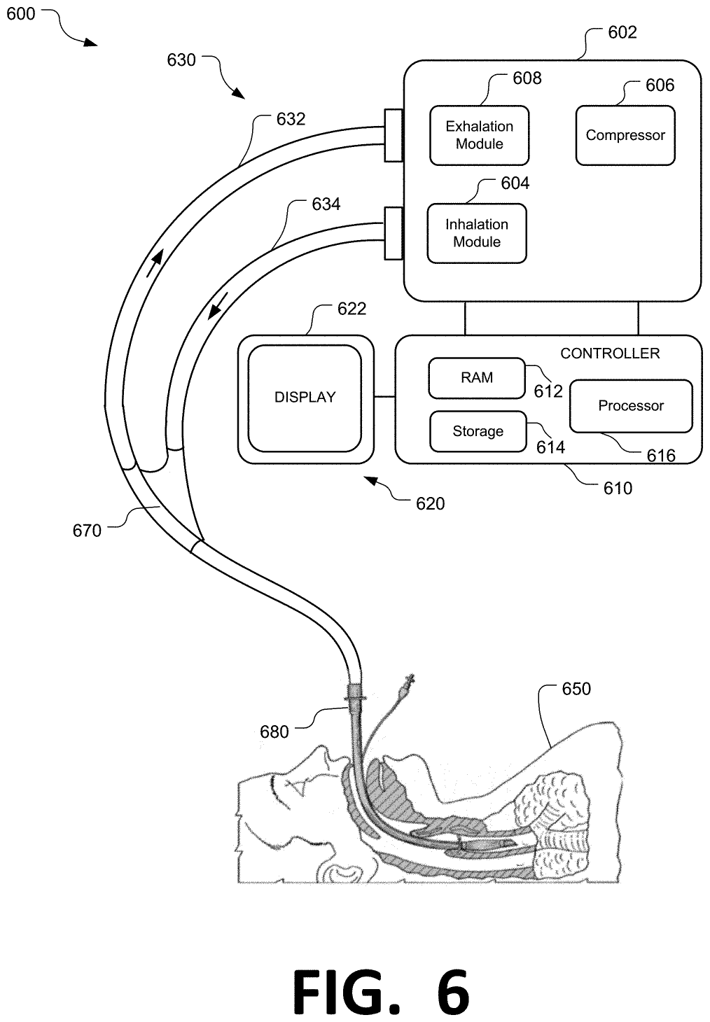

FIG. 6 is a diagram illustrating an example of a ventilator 600 connected to a human patient 650. Ventilator 600 includes a pneumatic system 602 (also referred to as a pressure generating system 602) for circulating breathing gases to and from patient 650 via the ventilation tubing system 630, which couples the patient to the pneumatic system via an invasive (e.g., endotracheal tube, as shown) or a non-invasive (e.g., nasal mask) patient interface.

Ventilation tubing system 630 may be a two-limb (shown) or a one-limb circuit for carrying gases to and from the patient 650. In a two-limb example, a fitting, typically referred to as a “wye-fitting” 670, may be provided to couple a patient interface 680 to an inhalation limb 634 and an exhalation limb 632 of the ventilation tubing system 630.

Pneumatic system 602 may have a variety of configurations. In the present example, system 602 includes an exhalation module 608 coupled with the exhalation limb 632 and an inhalation module 604 coupled with the inhalation limb 634. Compressor 606 or other source(s) of pressurized gases (e.g., air, oxygen, and/or helium) is coupled with inhalation module 604 to provide a gas source for ventilatory support via inhalation limb 634. The pneumatic system 602 may include a variety of other components, including mixing modules, valves, sensors, tubing, accumulators, filters, etc.

Controller 610 is operatively coupled with pneumatic system 602, signal measurement and acquisition systems, and an operator interface 620 that may enable an operator to interact with the ventilator 600 (e.g., change ventilator settings, select operational modes, view monitored parameters, etc.). Controller 610 may include memory 612, one or more processors 616, storage 614, and/or other components of the type found in command and control computing devices. In the depicted example, operator interface 620 includes a display 622 that may be touch-sensitive and/or voice-activated, enabling the display 622 to serve both as an input and output device.

The memory 612 includes non-transitory, computer-readable storage media that stores software that is executed by the processor 616 and which controls the operation of the ventilator 600. In an example, the memory 612 includes one or more solid-state storage devices such as flash memory chips. In an alternative example, the memory 612 may be mass storage connected to the processor 616 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 616. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

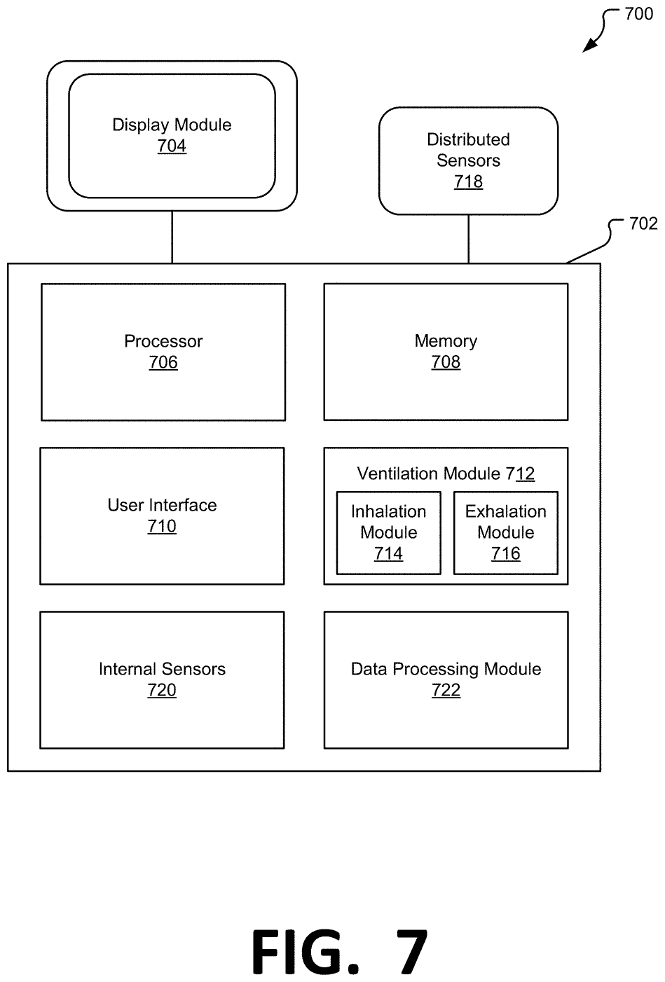

FIG. 7 is a block-diagram illustrating an example of a ventilatory system 700. Ventilatory system 700 includes ventilator 702 with its various modules and components. That is, ventilator 702 may further include, among other things, memory 708, one or more processors 706, user interface 710, and ventilation module 712 (which may further include an inhalation module 714 and an exhalation module 716). Memory 708 is defined as described above for memory 708. Similarly, the one or more processors 706 are defined as described above for one or more processors 706. Processors 706 may further be configured with a clock whereby elapsed time may be monitored by the system 700.

The ventilation system 700 may also include a display module 704 communicatively coupled to ventilator 702. Display module 704 provides various input screens, for receiving input, and various display screens, for presenting useful information. Inputs may be received from a clinician. The display module 704 is configured to communicate with user interface 710 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 702 may be provided, for instance by a wheel, keyboard, mouse, or other suitable interactive device. Thus, user interface 710 may accept commands and input through display module 704. Display module 704 may also provide useful information in the form of various ventilatory data regarding the physical condition of a patient and/or a prescribed respiratory treatment. The useful information may be derived by the ventilator 702, based on data collected by a data processing module 722, and the useful information may be displayed in the form of graphs, wave representations (e.g., a waveform), pie graphs, numbers, or other suitable forms of graphic display. For example, the data processing module 722 may be operative to determine ventilation settings (otherwise referred to as ventilatory settings, or ventilator settings) associated with oxygen-rich environments, leak detection, oxygen detection, release of one or more safety valves, etc., as detailed herein.

Ventilation module 712 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with an oxygen-rich environment. In some cases, certain ventilation settings may be adjusted based on the exhalation flow, e.g., to optimize the prescribed treatment.

Ventilation module 712 may further include an inhalation module 714 configured to deliver gases to the patient and an exhalation module 716 configured to receive exhalation gases from the patient, according to ventilation settings that may be based on the exhalation flow. As described herein, inhalation module 714 may correspond to the inhalation module 604 and 714, or may be otherwise coupled to source(s) of pressurized gases (e.g., air, oxygen, and/or helium), and may deliver gases to the patient. As further described herein, exhalation module 716 may correspond to the exhalation module 608 and 716, or may be otherwise coupled to gases existing the breathing circuit.

Although the present disclosure discusses the implementation of these techniques in the context of a ventilator with systems and methods for isolating gas leaks, the techniques introduced above may be implemented for a variety of medical devices or devices utilizing flammable gases or any other device at increased risk of fire. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Although this disclosure describes isolating components of a ventilator used to deliver concentrated oxygen to a patient, it should be appreciated that aspects described herein may be applied to a variety of pneumatic systems which have a risk of leaking any type of gas, including pneumatic flow mechanics, valves, flow sensing equipment, etc. Additionally, although aspects of the disclosure describe isolating concentrated oxygen, it should be appreciated that the disclosure may be applicable to isolating any gas from one or more systems (e.g., flammable gases such as oxygen, hydrogen, ammonia, methane, propane, etc., noxious gases such as bromine, chlorine, fluorine, phosphine, etc., or other gases not desirable in a system).

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces, and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A ventilator, comprising:
a housing of the ventilator, the housing comprising an exterior surface and an interior surface;
an internal oxygen line located inside the housing, the internal oxygen line configured to carry concentrated oxygen at a line pressure;
an orifice extending through the housing from the exterior surface to the interior surface; and
an isolation sleeve encasing the internal oxygen line, the isolation sleeve including:
a first end; and
a second end coupled to the orifice thereby allowing a gas inside the isolation sleeve to escape outside the housing.

2. The ventilator of claim 1, wherein the line pressure is between 15-50 PSI.

3. The ventilator of claim 2, wherein the gas inside the isolation sleeve is at atmospheric pressure.

4. The ventilator of claim 1, wherein the internal oxygen line has a first radius and the isolation sleeve has a second radius based on the first radius.

5. The ventilator of claim 1, wherein a material of the isolation sleeve is silicon.

6. The ventilator of claim 4, wherein the orifice has a third radius larger than the first radius and is located coaxially with the internal oxygen line.

7. The ventilator of claim 1, wherein the oxygen line is fluidly coupled to a ventilator component inside the housing.

8. The ventilator of claim 7, wherein the isolation sleeve also encases the ventilator component.

9. The ventilator of claim 8, wherein the isolation sleeve comprises a first sleeve segment associated with the oxygen line and a second sleeve segment associated with the ventilator component.

10. The ventilator of claim 9, wherein the second sleeve segment associated with the ventilator component is coupled to an isolation vent thereby allowing the gas inside the second sleeve segment of the isolation sleeve to escape outside the housing.

11. A ventilator, comprising:
a housing comprising an exterior surface and an interior surface;
an internal oxygen line located inside the housing, the internal oxygen line configured to carry concentrated oxygen at a line pressure;
a component located inside the housing, the component fluidly coupled with the oxygen line;
a first isolation sleeve encasing the internal oxygen line and comprising a first exhaust outside of the housing; and
a second isolation sleeve encasing the component and comprising a second exhaust outside of the housing.

12. The ventilator of claim 11, wherein the first exhaust comprises a first orifice and the second exhaust comprises a second orifice, wherein the first orifice and the second orifice extend from the exterior surface to the interior surface of the housing.

13. The ventilator of claim 12, wherein the first isolation sleeve is coupled to the first orifice.

14. The ventilator of claim 13, wherein the second isolation sleeve is coupled to the second orifice via an isolation vent.

15. The ventilator of claim 11, wherein the first isolation sleeve and the second isolation sleeve are fluidly coupled.

16. The ventilator of claim 15, wherein an isolation gas in the first isolation sleeve is isolated from the inside of the housing.

17. A ventilator, comprising:
a housing comprising an exterior surface and an interior surface;
an orifice having a first radius and extending through the housing from the exterior surface from the interior surface;
an oxygen line having a second radius fed through the orifice from the outside of the housing to the inside of the housing; and
an isolation sleeve inside the housing coupled to the orifice, the isolation sleeve having the first radius and encasing a portion of the oxygen line existing inside the housing.

18. The ventilator of claim 17, wherein an isolation gas inside the isolation sleeve is fluidly coupled with an outside gas outside the housing, and wherein the isolation gas is isolated from an inside gas inside the housing.

19. The ventilator of claim 17, wherein the oxygen line is coupled to an oxygen source.

20. The ventilator of claim 17, wherein the first radius is based on the second radius.
